(19) United States
(12) Patent Application Publication
(10) Pub. No.: US 2025/0295704 A1
(43) Pub. Date: Sep. 25, 2025

(54) OSTEOARTHRITIS TREATMENT COMPOSITION USING iPSC-DERIVED MITOCHONDRIA

(71) Applicant: THE CATHOLIC UNIVERSITY OF KOREA INDUSTRY-ACADEMIC COOPERATION FOUNDATION, Seoul (KR)

(72) Inventors: Mi-La CHO, Seoul (KR); Seok-Jung KIM, Seoul (KR); A Ram LEE, Jinju-si (KR); Joo-Yeon JHUN, Seoul (KR); Se Gyeong HAN, Namwon-si (KR); Jeong Su LEE, Seoul (KR); Hyun-Sik NA, Seoul (KR); Byung Ha CHUNG, Seoul (KR); Hanbin LEE, Seongnam-si (KR); Sun Woo LIM, Seoul (KR); Yoo-Jin SHIN, Seoul (KR); Seon-Yeong LEE, Seoul (KR)

(73) Assignee: THE CATHOLIC UNIVERSITY OF KOREA INDUSTRY-ACADEMIC COOPERATION FOUNDATION, Seoul (KR)

(21) Appl. No.: 19/086,575

(22) Filed: Mar. 21, 2025

(30) Foreign Application Priority Data
Mar. 22, 2024 (KR) 10-2024-0039853

(51) Int. Cl. A61K 35/545; A61P 19/02

(52) U.S. Cl. CPC A61K 35/545; A61P 19/02

(57) ABSTRACT

Provided is a composition for the treatment of osteoarthritis using iPSC-derived mitochondria. Therefore, it was confirmed that the iPSC-derived mitochondria of the present disclosure increased the transplant ability into chondrocytes, suppressed the expression of the MMP series, which were cartilage metabolic factors, and suppressed the death of chondrocytes. In addition, it was confirmed that the iPSC-derived mitochondria suppressed damage to joint tissue in an osteoarthritis animal model, reduced immune cells, and increased immune osteoarthritis-causing regulatory cells. In addition, it was confirmed that the expression of UCP2 in iPSC-derived mitochondria was significantly increased compared to mitochondria isolated from other origin cells, and that UCP2 was closely related to the treatment effect of osteoarthritis caused by mitochondrial transplantation. In addition, it was confirmed that iPSC-derived mitochondria overexpressing UCP2 improves osteoarthritis as a cartilage regeneration effect.

### CROSS-REFERENCE TO RELATED APPLICATIONS

This application claims the priority of Korean Patent Application No. 10-2024-0039853 filed on Mar. 22, 2024, in the Korean Intellectual Property Office, the disclosure of which is incorporated herein by reference.

### BACKGROUND

### Field

The present disclosure relates to a composition for the treatment of osteoarthritis using iPSC-derived mitochondria.

### Description of the Related Art

Osteoarthritis is a type of arthritis, also called degenerative arthritis, and refers to arthritis caused by degenerative changes in cartilage and marginal bone in synovial joint. In other words, osteoarthritis is a disease characterized by gradual loss of articular cartilage, hypertrophy of the bone located below the cartilage, bone formation at the joint margin, and non-specific synovial inflammation. Osteoarthritis is a disease caused by damage to cartilage due to aging or excessive physical pressing (e.g., obesity, trauma, etc.). Accordingly, osteoarthritis causes severe pain and movement disorders in joints that bear a lot of weight, such as the knee joint and hip joint, and lead to joint deformation if left for a long time.

Osteoarthritis progresses through the following stages: a cartilage change stage (stage 1), in which the water content in the cartilage increases to cause swelling; a fibrillation stage (stage 2), in which the cartilage surface is cracked and torn and then damaged by the destruction of the cartilage to expose the bone and narrow the joint cavity; a chondrocyte reduction stage (stage 3), in which chondrocytes begin to produce cartilage to restore the cartilage, but cartilage destruction occurs faster than cartilage production to reduce the chondrocytes; a bone deformation stage (stage 4), in which the bone is deformed to cause joint deformity and dysfunction; and a joint soft tissue change stage (stage 5), in which soft tissue is thickened.

Rheumatoid arthritis, which is classified differently from osteoarthritis, is a chronic autoimmune disease characterized by inflammation and proliferation of synovial cells, and unlike osteoarthritis, osteoporosis and bone erosion occur in the bones around the joints. Rheumatoid arthritis progresses through a stage in which inflammation of the synovial membrane spreads to the joint capsule, ligament, and tendon (stage 1), a stage in which gaps between joints narrow due to gradual destruction of the joint cartilage and the tension of the joint capsule and the ligament is lost (stage 2), a stage in which inflammation invades the bone and partial erosion occurs in the bone (stage 3), and a stage in which joint function is lost (stage 4). Therefore, osteoarthritis and rheumatoid arthritis have completely different causes and stages of progression, and also have different treatment methods thereof.

Currently, drugs such as acetaminophen, tramadol, nonsteroidal antiinflammatory drugs (NSAIDs), diacerein, and glucosamine have been used to treat osteoarthritis. Among these drugs, nonsteroidal anti-inflammatory drugs are pointed out as problematic due to gastrointestinal side effects such as gastric and duodenal ulcers. Therefore, when administering the drugs to patients with osteoarthritis who have risk factors for gastrointestinal side effects, cytoprotective agents such as rebamipide, H2-receptor antagonists such as cimetidine and ranitidine, and proton pump inhibitors such as omeprazole are prescribed at the same time.

Meanwhile, mitochondria are organelles of eukaryotic cells which are involved in the synthesis and regulation of adenosine triphosphate (ATP), which is an energy source within cells. The mitochondria are associated with various metabolic pathways in the body, such as cell signaling, cell differentiation, and cell death, as well as control of the cell cycle and cell growth. The mitochondria are organelles that have their own genome and play a central role in cellular energy metabolism. The mitochondria produce energy through electron transfer and oxidative phosphorylation processes, and play an important role in the cell death signaling pathway.

It has been reported that decreased energy production due to a decreased mitochondrial function causes various diseases. When the function of an electron transfer chain reaction is reduced due to mutations in the mitochondrial genome and protein, decreased ATP production, excessive production of reactive oxygen, reduced calcium regulation function, and the like occur. In this case, changes in the membrane permeability of mitochondria occur to cause an abnormal cell death function and lead to cancer and incurable diseases.

As such, human diseases caused by mitochondrial dysfunction have been reported to include mitochondrial-related genetic diseases, diabetes, heart disease, senile dementia such as Parkinson's disease or Alzheimer's disease, and the occurrence and metastasis of various cancers. In addition, the common features found in at least 200 different types of cancer consist of impairment of apoptosis functions, increased inflammatory response, and increased abnormal metabolic activity. In addition, research has currently been conducted on the relationship between mitochondria and various diseases.

Accordingly, the present inventors confirmed that iPSC-derived mitochondria could improve osteoarthritis and the iPSC-derived mitochondria have increased mitochondrial activity, and then completed the present disclosure.

### SUMMARY

An object to be achieved by the present disclosure is to provide a pharmaceutical composition for the prevention or treatment of osteoarthritis, including induced pluripotent stem cell (iPSC)-derived mitochondria as an active ingredient.

Another object to be achieved by the present disclosure is to provide a pharmaceutical composition for cartilage regeneration, including iPSC-derived mitochondria as an active ingredient.

Objects of the exemplary embodiment of the present disclosure are not limited to the above-mentioned objects, and other objects, which are not mentioned above, may be clearly understood by those skilled in the art from the following descriptions.

An aspect of the present disclosure provides a pharmaceutical composition for the prevention or treatment of osteoarthritis, including induced pluripotent stem cell (iPSC)-derived mitochondria as an active ingredient.

Further, another aspect of the present disclosure provides a pharmaceutical composition for cartilage regeneration, including iPSC-derived mitochondria as an active ingredient.

Therefore, it was confirmed that the iPSC-derived mitochondria of the present disclosure increased the transplant ability into chondrocytes, suppressed the expression of the MMP series, which were cartilage metabolic factors, and suppressed the death of chondrocytes. In addition, it was confirmed that the iPSC-derived mitochondria suppressed damage to joint tissue in an osteoarthritis animal model, reduced osteoarthritis-causing immune cells, and increased immune regulatory cells. In addition, it was confirmed that the expression of UCP2 in iPSC-derived mitochondria was significantly increased compared to mitochondria isolated from other origin cells, and that UCP2 was closely related to a treatment effect of osteoarthritis caused by mitochondrial transplantation. In addition, iPSC-derived mitochondria overexpressing UCP2 can improve osteoarthritis by a cartilage regeneration effect, and thus can be usefully utilized in related industries.

The effects of the present disclosure are not limited to the aforementioned effects, and other effects, which are not mentioned above, will be apparently understood to a person having ordinary skill in the art from the following description.

The objects to be achieved by the present disclosure, the means for achieving the objects, and the effects of the present disclosure described above do not specify essential features of the claims, and, thus, the scope of the claims is not limited to the disclosure of the present disclosure.

### BRIEF DESCRIPTION OF THE DRAWINGS

The above and other aspects, features and other advantages of the present disclosure will be more clearly understood from the following detailed description taken in conjunction with the accompanying drawings, in which:

FIG. 1 is a diagram showing the purification quality and delivery efficiency into chondrocytes of iPSC-derived mitochondria of the present disclosure (A: confirmation of mitochondrial quality, B: delivery efficiency and quantification);

FIG. 2 is a diagram showing an effect of iPSC-derived mitochondria of the present disclosure on regulation of the expression of cartilage metabolic factors (A: quantification of qRT-PCR results, B: quantification of ELISA results);

FIG. 3 is a diagram showing the expression of inflammatory apoptotic factors in iPSC-derived mitochondria of the present disclosure analyzed by Western blot (A: Western blot results, B: quantification of analysis results);

FIG. 4 is a diagram showing an osteoarthritis improvement effect of iPSC-derived mitochondria of the present disclosure confirmed by pain analysis (A: schematic diagram of animal model fabrication process, B: quantification of pain analysis results);

FIG. 5 is a diagram showing a cartilage damage protection effect of iPSC-derived mitochondria of the present disclosure analyzed by histology (A: safranin O staining and quantification, B: immunohistochemical staining and quantification);

FIG. 6 is a diagram showing flow cytometry of immune cell subtypes in an osteoarthritis animal model according to the administration of iPSC-derived mitochondria of the present disclosure;

FIG. 7 is a diagram showing the expression of UCP2 according to a cell type analyzed by Western blot (A: Western blot results, B: UCP2 expression quantification);

FIG. 8 is a diagram showing the expression of mitochondrial UCP2 according to an origin cell analyzed by Western blot (A: Western blot results, B: comparison of cytoplasmic and mitochondrial UCP2 expression);

FIG. 9 is a diagram showing an effect of UCP2 siRNA iPSC-derived mitochondria on reducing immune cell regulation to confirm the function of iPSC-derived mitochondria according to UCP2 expression (A: confirmation of UCP2 expression, B: immune cell quantification);

FIG. 10 is a diagram showing an effect of UCP2-overexpressed Muscle-derived mitochondria on suppressing cartilage metabolism factors to confirm the function of iPSC-derived mitochondria according to UCP2 expression (A: confirmation of UCP2 expression, B: quantification of cartilage metabolism factor and inflammatory factor expression);

FIG. 11 is a diagram showing an effect of UCP2-overexpressed Muscle-derived mitochondria on increasing cartilage regeneration factors;

FIG. 12 is a diagram comparing an inflammatory cytokine regulation effect according to treatment of iPSC-derived mitochondria of the present disclosure and an osteoarthritis therapeutic agent;

FIG. 13 is a diagram comparing the expression of cartilage metabolic factors and inflammatory factors according to treatment of iPSC-derived mitochondria of the present disclosure and an osteoarthritis therapeutic agent; and

FIG. 14 is a diagram comparing the expression of cartilage regeneration factors according to treatment of iPSC-derived mitochondria of the present disclosure and an osteoarthritis therapeutic agent.

### DETAILED DESCRIPTION OF THE EMBODIMENT

Hereinafter, exemplary embodiments of the present disclosure will be described in detail with reference to the accompanying drawings. In the following description, detailed descriptions of techniques well-known to those skilled in the art may be omitted. Further, in describing the present disclosure, the detailed description of associated known functions or configurations will be omitted if it is determined to unnecessarily make the gist of the present disclosure unclear. In addition, terminologies used herein are terminologies used to properly express preferred exemplary embodiments of the present disclosure, which may vary according to a user, an operator's intention, or customs in the art to which the present disclosure pertains.

Accordingly, definitions of the terminologies need to be described based on contents throughout this specification. Throughout this specification, unless explicitly described to the contrary, when a certain part “comprises” a certain component, it will be understood to imply the inclusion of stated elements, not the exclusion of any other elements.

The present disclosure provides a pharmaceutical composition for the prevention or treatment of osteoarthritis, including induced pluripotent stem cell (iPSC)-derived mitochondria as an active ingredient.

As used in the present disclosure, the term “prevention” refers to any action that suppresses the symptoms of a specific disease or delays its progression by administering the composition of the present disclosure.

As used in the present disclosure, the term “treatment” refers to any action that improves or beneficially changes the symptoms of a specific disease by administering the composition of the present disclosure.

The pharmaceutical composition of the present disclosure may further include an adjuvant in addition to the active ingredient. The adjuvant may be used with any adjuvant known in the art without limitation, but further include, for example, a Freund's complete adjuvant or an incomplete adjuvant to increase the effect thereof.

The pharmaceutical composition according to the present disclosure may be prepared in the form of incorporating the active ingredient into a pharmaceutically acceptable carrier. Here, the pharmaceutically acceptable carrier includes carriers, excipients and diluents commonly used in a pharmaceutical field. The pharmaceutically acceptable carrier that may be used in the pharmaceutical composition of the present disclosure is not limited thereto, but may include lactose, dextrose, sucrose, sorbitol, mannitol, xylitol, erythritol, maltitol, starch, acacia gum, alginate, gelatin, calcium phosphate, calcium silicate, cellulose, methyl cellulose, polyvinylpyrrolidone, water, methyl hydroxybenzoate, propyl hydroxybenzoate, talc, magnesium stearate, and mineral oil.

The pharmaceutical composition of the present disclosure may be formulated and used in the form of oral formulations, such as powders, granules, tablets, capsules, suspensions, emulsions, syrups, aerosols, etc., external preparations, suppositories, or sterile injectable solutions according to each conventional method.

The formulations may be prepared by using diluents or excipients, such as a filler, an extender, a binder, a wetting agent, a disintegrating agent, a surfactant, etc., which are generally used. Solid formulations for oral administration include tablets, pills, powders, granules, capsules, etc., and these solid formulations may be prepared by mixing at least one or more excipients, for example, starch, calcium carbonate, sucrose, lactose, gelatin, etc. with the active ingredient. Further, lubricants such as magnesium stearate and talc may be used in addition to simple excipients. Liquid formulations for oral administration may correspond to suspensions, oral liquids, emulsions, syrups, etc., and may include various excipients, for example, a wetting agent, a sweetener, an aromatic agent, a preserving agent, etc., in addition to the commonly used diluents, such as water and liquid paraffin. Formulations for parenteral administration include sterile aqueous solutions, non-aqueous solvents, suspensions, emulsions, lyophilized agents, and suppositories. As the non-aqueous solvent and the suspension, propylene glycol, polyethylene glycol, vegetable oil such as olive oil, injectable ester such as ethyl oleate, etc. may be used. As the base material of the suppository, witepsol, Tween 61, cacao butter, laurinum, glycerogelatin, etc. may be used.

The pharmaceutical composition according to the present disclosure may be administered to a subject through various routes. All methods of administration may be expected, and the pharmaceutical composition may be administered by, for example, oral, intravenous, intramuscular, subcutaneous, and intraperitoneal injection.

The dose of the pharmaceutical composition according to the present disclosure is selected in consideration of the age, body weight, sex, physical conditions, and the like of a subject. It is obvious that the concentration of the active ingredient included in the pharmaceutical composition may be variously selected according to a subject, and preferably included in the pharmaceutical composition at a concentration of 0.01 to 5,000 μg/ml. When the concentration is less than 0.01 μg/ml, pharmaceutical activity may not be exhibited, and when the concentration exceeds 5,000 μg/ml, toxicity to the human body may be exhibited.

The “induced pluripotent stem cell (iPSC)” of the present disclosure is a pluripotent stem cell that may be directly produced from adult cells. The iPSC is converted into induced pluripotent stem cells by expressing a specific transcription factor gene in adult somatic cells to reprogram the cells. The key to producing iPSC is which reprogramming factors are used, and representative reprogramming factors are a “Yamanaka factor” used in a method announced by the Yamanaka research team, and transcription factors Oct4, Sox2, cMyc, and Klf4. Through continuous follow-up research on the reprogramming factors, not only transcription factors but also new regulatory factors such as miRNA have been discovered. The method for producing iPSC includes (1) isolating and culturing necessary cells from adults, and then (2) introducing a gene capable of inducing stem cells into the isolated cells using a viral vector. (3) Thereafter, cells expressing the introduced gene are labeled with an embryonic stem cell marker Fbx15 and isolated using antibiotics. Then, the isolated cells are cultured on a support cell layer according to a stem cell culture method. (4) In the cultured cells, some of the transformed cells are reprogrammed into iPSCs, and colonies similar to embryonic stem cells are selected to produce iPSCs.

The “mitochondria” of the present disclosure is one of the cell organelles, and involved in cellular respiration, and cells with active respiration contain many mitochondria. The mitochondria are surrounded by double films and the inside thereof is made of a winding inner membrane called cristae. DNA and RNA exist in mitochondria, and the mitochondria are 0.2 to 3 μm in size and involved in cellular respiration. The most important function of mitochondria serves to synthesize ATP as an energy source, through food taken into the body. In the inner membrane of the mitochondria, there is a protein called ‘ATP synthase’, which serves to produce ATP. Hydrogen ions formed between the inner and outer membranes of the mitochondria are introduced into the inner membrane of the mitochondria through food, and phosphate and ADP (a form in which two phosphates bind to adenosine) bind to each other by ATP synthase to produce ATP (a form in which three phosphates bind to adenosine). In addition, the mitochondria also play a role in killing cells of which the function is lost, which is called apoptosis, and the mitochondria absorb cells with damaged DNA or fragment the DNA, leading to apoptosis. In addition, the mitochondria also prevent cells of which the function has been already lost from mutating into cancer cells or other cells. The mitochondria are structures that play a central role in controlling respiration, and are also involved in oxidation of pyruvate and acetyl COA, in which sugars are produced by the catabolism of lipids, and oxidative phosphorylation through an electron transport chain. In this way, the mitochondria play a central role in the oxidation of sugars and fatty acids and the oxidative phosphorylation accompanying the oxidation.

According to an exemplary embodiment of the present disclosure, mitochondria may be mitochondria that overexpress uncoupling protein 2 (UCP2).

The “uncoupling protein 2 (UCP2)” of the present disclosure is mitochondrial uncoupling protein 2, a protein belonging to the mitochondrial anion transfer protein family. The UCP uncouples oxidative phosphorylation from ATP synthesis by dissipating mitochondrial membrane potential as heat, called mitochondrial proton leak. The UCP promotes the transfer of protons from the inner mitochondrial membrane to the outer mitochondrial membrane, and reduces the mitochondrial membrane potential of mammalian cells to reduce the production of reactive oxygen species. Unlike UCP1 and UCP3, which are mainly expressed in adipose and smooth muscles, UCP2 is expressed in various tissues including kidney, liver, gastrointestinal tract, brain, and skeletal muscle, and the exact mechanism of UCP2 is unknown, but it is known that its main function is related to the control of mitochondria-derived reactive oxygen species.

According to an exemplary embodiment of the present disclosure, the mitochondria may suppress the expression of cartilage metabolic factors, and the cartilage metabolic factor may be a factor selected from the group consisting of MMP3, MMP9, MMP13, and MCP1.

According to an exemplary embodiment of the present disclosure, the mitochondria may suppress the death of chondrocytes, and the suppressing of the death of chondrocytes may be suppressing phosphorylation of RIP3 or MLKL.

According to an exemplary embodiment of the present disclosure, the mitochondria may regulate immune cells, and the immune cell may be Th1, Th2, Th17 or Treg.

According to an exemplary embodiment of the present disclosure, the regulating of the immune cells may be suppressing Th1, Th2 or Th17.

According to an exemplary embodiment of the present disclosure, the regulating of the immune cells may be increasing Treg.

According to an exemplary embodiment of the present disclosure, the mitochondria may increase the expression of cartilage regeneration factors, and the cartilage regeneration factor may be selected from the group consisting of transcription factor SOX9 (SOX9), Runt-related transcription factor 2 (RUNX2), Aggrecan, and Type II collagen.

The “transcription factor SOX9 (SOX9)” of the present disclosure is a central transcription factor in cartilage, and is a gene that is expressed in multipotent skeletal progenitor cells to be activated throughout the differentiation of chondrocytes.

The “Runt-related transcription factor 2 (RUNX2)” of the present disclosure is a gene that regulates osteoblast differentiation and chondrocyte maturation, and the RUNX2 regulates cartilage formation by regulating the transcription of NELL1, a key functional mediator of cartilage formation.

The “Aggrecan” of the present disclosure is an essential component of the extracellular matrix in cartilage tissue including a growth plate, and is a protein that has resistance to pressure in cartilage.

The “Type II collagen” of the present disclosure is a hyaline cartilage component including articular cartilage of a joint surface, and the Type II collagen is collagen that enables cartilage to contain proteoglycan aggregates and provides tensile strength to cartilage tissue.

Further, the present disclosure provides a pharmaceutical composition for cartilage regeneration, including iPSC-derived mitochondria as an active ingredient.

Hereinafter, the present disclosure will be described in more detail through Examples. These Examples are to explain the present disclosure in more detail, and it will be apparent to those skilled in the art that the scope of the present disclosure is not limited to these Examples.

### <Preparation Example 1> Isolation of iPSC-Derived Mitochondria

In order to confirm whether the induced pluripotent stem cell (iPSC)-derived mitochondria of the present disclosure had an effect of improving osteoarthritis, induced pluripotent stem cells were isolated from iPSCs. Mitochondria were isolated from a WTC11 cell line as a human-derived iPSC cell line, using a Mitochondria Isolation Kit (Thermo, Waltham, MA, USA; #89874). Specifically, cells were obtained, added with Reagent A of the kit, vortexed for 5 seconds, and then iced for 2 minutes. Thereafter, the cells were added with Reagent B, vortexed for 5 seconds, and iced for 5 minutes. Thereafter, the cells were vortexed every minute for 5 minutes and then mixed with Reagent C. Thereafter, the cells were centrifuged under conditions of 700×g and 4° C. for 10 minutes to obtain the supernatant, centrifuged under conditions of 12,000×g and 4° C. for 10 minutes to remove the supernatant, and the pellet was added with Reagent C and resuspended, centrifuged under conditions of 10,000×g and 4° C. for 10 minutes to remove the supernatant and obtain mitochondria. 8 to 10 μg of mitochondria was obtained at a concentration of 1×10⁶ cells of the WTC11 cell line.

### <Example 2> Confirmation of Effect of iPSC-Derived Mitochondria on Regulating Cartilage Metabolism Factors

In order to confirm whether the iPSC-derived mitochondria of the present disclosure improved osteoarthritis, the purification quality and chondrocyte transplantation efficiency of mitochondria were analyzed to confirm an effect of regulating cartilage metabolism factors. Specifically, in order to measure the quality of mitochondria after isolating the iPSC-derived mitochondria, the protein expression of tubulin as a cytosolic factor and COX4 as a mitochondrial factor were analyzed by Western blotting. Thereafter, the mitochondria isolated from chondrocytes were stained with MTDR, and the delivery efficiency into chondrocytes according to the amount of mitochondria (0.5 or 1 μg) was analyzed by flow cytometry. As control groups, a Nil group, which was an untreated control group, and a Vehicle group, which was treated with only the same amount of solvent, were used.

Thereafter, chondrocytes obtained from mice were stimulated with IL-1β, and then treated with 1 ng of iPSC-derived mitochondria, and co-cultured. As control groups, a Nil group, which was an untreated control group, and a Vehicle group, which was treated with only the same amount of solvent instead of mitochondria, were used. After 1 day of culture, cells were harvested, and the expression of cartilage metabolic factors MMP3, MMP9, and MMP13 was analyzed by quantitative PCR (qRT-PCR), and the expression of a cartilage metabolic factor MCP1 in the culture medium was analyzed by ELISA.

In addition, after 2 days of culture, the cells were harvested and the protein expression and phosphorylation of inflammatory apoptotic factors RIP3 and MLKL were analyzed by Western blot.

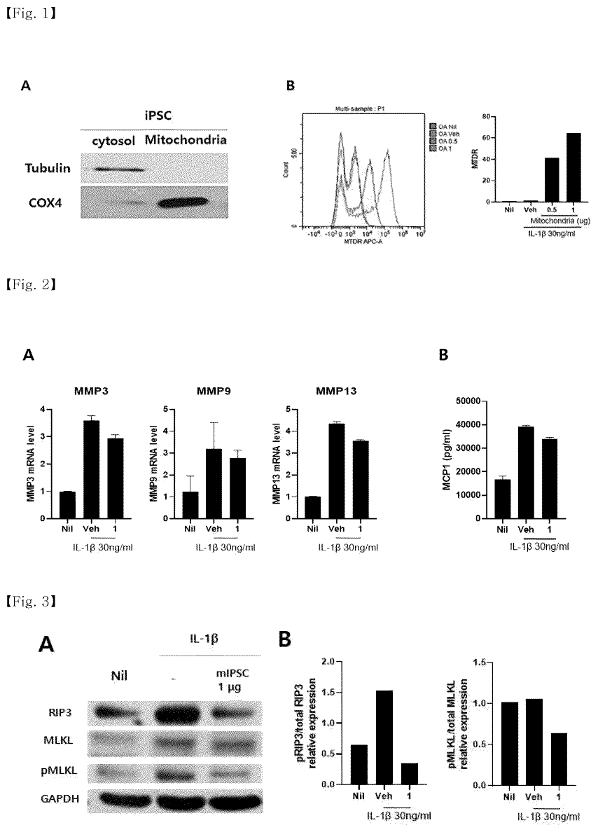

As a result of confirming the quality of mitochondria, as shown in A of FIG. 1, it was confirmed that iPSC-derived mitochondria were well isolated, the protein expression of tubulin and COX4 was confirmed, and 1 μg of iPSC was transferred per 1×10⁵ chondrocytes, so that 60% of iPSC-derived mitochondria were transferred (B of FIG. 1).

In addition, as shown in A of FIG. 2, compared to the Nil group, in the Vehicle group, the expression of cartilage metabolic factors MMP3, MMP9, and MMP13 was significantly increased, but in the group treated with iPSC-derived mitochondria, the increased expression of cartilage metabolic factors was significantly decreased. In addition, it was confirmed that the expression of the cartilage metabolic factor MCP1 in the culture medium was also significantly decreased (B of FIG. 2).

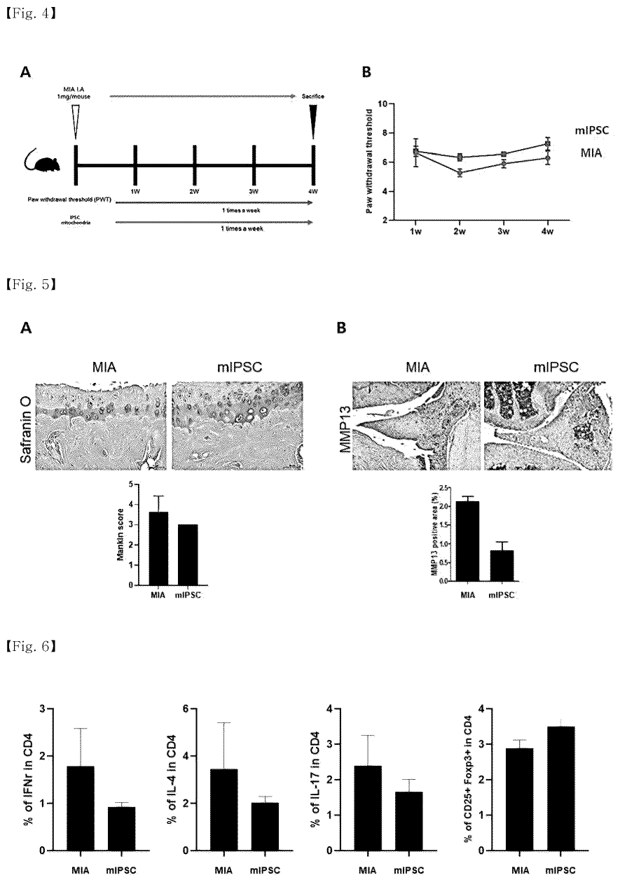

In addition, as a result of confirming the protein expression and phosphorylation of inflammatory apoptotic factors on day 2 of culture, it was confirmed that compared to the Nil group, in the Vehicle group, the expression of the inflammatory significantly increased, and the apoptotic factor RIP3 was expression and phosphorylation of MLKL were increased, but when iPSC-derived mitochondria were treated, both the increased expression of RIP3 and the expression and phosphorylation of MLKL were significantly decreased (FIG. 3).

### <Example 3> Confirmation of Improvement of Osteoarthritis by iPSC-Derived Mitochondria

In order to confirm whether the iPSC-derived mitochondria of the present disclosure improved osteoarthritis, an improvement effect in a MIA-induced osteoarthritis animal model was confirmed. Specifically, 6-week-old male Wistar rats were bred at a temperature of 21 to 22° C. in a light-dark cycle at 12-hour intervals and were raised by supplying sterilized water and Thereafter, a 3 mg/50 μl dose of monosodium iodoacetate feed. (MIA, Sigma, ST. Louis, MO) was administered to the right knee of rats to induce osteoarthritis. MIA was dissolved in physiological saline and administered. Thereafter, the rats were divided into an MIA group that was not administered with any drug, but administered with a drug excipient, and a mIPSC group that was administered with the iPSC-derived mitochondria of the present disclosure into the joint cavity. The specific drug administration and the experimental schedule were shown in A of FIG. 4. Thereafter, pain due to osteoarthritis was measured using a dynamic plantat aesthesiometer (Ugo Basile, Comerio, Italy). A pain measurement method of a machine was performed by placing a mesh plate on a measuring machine, adding the rat in an acrylic animal fixation frame thereon, and then sticking the right paw injected with the drug using the measuring machine. After sticking, a time (s, seconds) taken by the machine to automatically remove the paw and any amount of weight (g) required to remove the paw were measured, and the time and weight at that time were confirmed. By using the time and weight, the degree of pain was measured using a paw withdrawal latency.

As a result, as shown in B of FIG. 4, it was confirmed that compared to the MIA group, in the group administered iPSC-derived mitochondria, the pain was decreased, and the paw withdrawal latency was increased.

### <Example 4> Confirmation of Cartilage Protection Effect of iPSC-Derived Mitochondria

In order to confirm whether the iPSC-derived mitochondria of the present disclosure improved osteoarthritis, cartilage tissue damage was confirmed. Specifically, the animal model of each group in Example 3 above was humanely sacrificed at the end of the experiment, and cartilage tissue was obtained and sectioned. Thereafter, the sectioned cartilage tissue was stained with Safranin O to confirm the Mankin score of cartilage tissue damage, and the expression of MMP3 in cartilage was confirmed by immunohistochemical staining.

As a result, as shown in FIG. 5, it was confirmed that compared to the MIA group, in the group administered with iPSC-derived mitochondria, the Mankin score was significantly reduced and the expression of MMP13 in cartilage tissue was significantly reduced.

### <Example 5> Confirmation of Regulation of Immune Cells in Osteoarthritis by iPSC-Derived Mitochondria

It was confirmed whether the iPSC-derived mitochondria of the present disclosure regulated immune cells in osteoarthritis. Specifically, spleen tissues were obtained from the mice sacrificed in Example 3 above, and T cell subtypes were analyzed by flow cytometry in cells isolated from the spleen tissues.

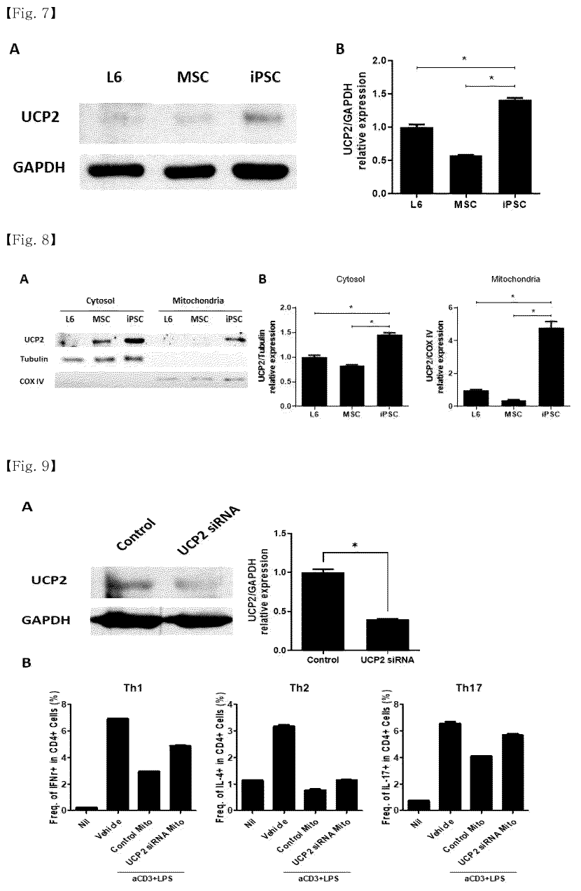

As a result, as shown in FIG. 6, it was confirmed that in the MIA group, the expression of Th1, Th2, and Th17 cells, which were autoimmune diseases-causing immune cells, was significantly increased, but in the group administered with iPSC-derived mitochondria, the increased expression of Th1, Th2, and Th17 was significantly decreased, and the expression of Treg, which was an immune regulatory cell, was significantly increased.

### <Example 6> Confirmation of Characteristics of iPSC-Derived Mitochondria

### <6-1> Confirmation of UCP2 Expression According to Cell Type

The expression of UCP2 in the iPSCs of the present disclosure and muscle cells or mesenchymal stem cells was compared. Specifically, proteins were extracted from iPSCs, and a muscle cell line L6 and mesenchymal stem cells (MSC), and the expression of UCP2 was confirmed by Western blotting.

As a result, as shown in FIG. 7, it was confirmed that the iPSCs of the present disclosure overexpressed UCP2 more than the muscle cell line L6 and MSC cells.

### <6-2> Confirmation of Mitochondrial UCP2 Expression According to Origin Cell

It was confirmed whether the iPSC-derived mitochondria of the present disclosure had a difference in UCP2 expression depending on an origin cell. Specifically, mitochondria were isolated from iPSCs, a muscle cell line L6, and MSC cells, and the expression of UCP2 in the mitochondria themselves was analyzed by Western blotting.

As a result, as shown in FIG. 8, in the isolated mitochondria, it was confirmed that there was a difference in the expression of UCP2 depending on an origin cell, and it was confirmed that the expression of UCP2 was significantly high in the iPSC-derived mitochondria of the present disclosure, and thus it was confirmed that the protein phenotype of the mitochondria varied depending on a cell line.

<6-3> Confirmation of Activity of UCP2 Using UCP2 siRNA

In Example 6-1 above, it was confirmed that UCP2 was overexpressed in the iPSC, thereby confirming whether the expression of UCP2 affected the function of the mitochondria and the regulation of immune cells. Specifically, the expression of UCP2 was reduced using UCP2 siRNA in the iPSC. Thereafter, mitochondria were obtained from the iPSC with reduced UCP2, and the obtained mitochondria were treated with mouse spleen cells, and then the distribution of immune cells was analyzed by flow cytometry. As a control group (Control), iPSCs that were not treated with UCP2 siRNA were used.

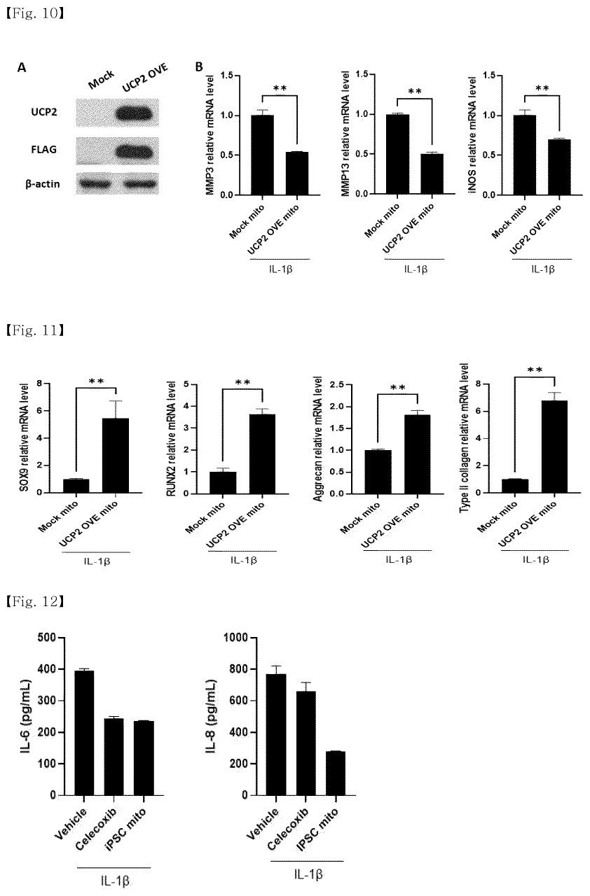

As a result, as shown in A of FIG. 9, it was confirmed that the expression of UCP2 in iPSC was reduced due to UCP2 siRNA.

In addition, it was confirmed that the iPSC-derived mitochondria with reduced expression of UCP2 reduced a frequency inhibition effect of Th1, Th2, Th17, and which were osteoarthritis pathogenic immune cells, and thus Th1, Th2, and Th17 cells increased compared to the Control group (B of FIG. 9), and thus it was confirmed that UCP2 affected an immune regulation effect of iPSC-derived mitochondria.

<6-4> Confirmation of Cartilage Metabolism Factor Inhibition Effect of Mitochondria Derived from UCP2-Overexpressing Muscle Cells

In Example 6-1 above, it was confirmed that UCP2 was overexpressed in iPSC, thereby confirming whether the expression of UCP2 affected the function of the mitochondria and the regulation of immune cells. Specifically, mitochondria (UCP2 OVE) were obtained from transformed muscle cells by injecting a vector overexpressing UCP2. Thereafter, chondrocytes obtained from mice were stimulated with IL-1B, and then treated with 1 ng of UCP2 OVE mitochondria, and co-cultured. As a control group, mitochondria derived from muscle cells transformed with a mock vector of UCP2 were used. Thereafter, the expression of cartilage metabolic factors MMP3 and MMP13 and iNOS in the inflammatory response was analyzed by quantitative PCR (qRT-PCR).

In addition, in order to confirm whether UCP2 OVE mitochondria promoted cartilage regeneration (chondrogenesis), the expression of transcription factor SOX9 (SOX9), Runt-related transcription factor 2 (RUNX2), Aggrecan, and Type II collagen, which were cartilage regeneration factors, was analyzed by quantitative PCR (qRT-PCR).

As a result, as shown in A of FIG. 10, it was confirmed that UCP2 was actually overexpressed in a UCP2 OVE group transfected with the UCP2 vector. In addition, it was confirmed that UCP2 OVE iPSC-derived mitochondria significantly reduced the expression of cartilage metabolism factors MMP3 and MMP13 and decreased iNOS compared to a Mock group (B of FIG. 10), and thus it was confirmed that the expression of UCP2 affected an osteoarthritis improvement effect of mitochondria.

In addition, as shown in FIG. 11, it was confirmed that UCP2 OVE iPSC-derived mitochondria increased the expression of cartilage regeneration factors SOX9, RUNX2, Aggrecan, and Type II collagen compared to the Mock group, and thus it was confirmed that mitochondrial UCP2 overexpression induced cartilage regeneration.

### <Example 7> Comparison of Effects of iPSC-Derived Mitochondria and Osteoarthritis Therapeutic Agents

It was confirmed that the iPSC-derived mitochondria of the present disclosure improved osteoarthritis, and thus an inflammatory cytokine regulation effect was compared with that of a conventional osteoarthritis therapeutic agent, Celecoxib. Specifically, chondrocytes obtained from mice were stimulated with IL-1β, and then treated with 1 ng of iPSC-derived mitochondria, and co-cultured (iPSC mito group). As control groups, a Vehicle group treated with the same amount of solvent and a Celecoxib group treated and cultured with 10 UM of the osteoarthritis therapeutic agent Celecoxib were used. After the culture was completed, the supernatant was obtained and the amounts of inflammatory cytokines IL-6 and IL-8, which were secreted into the supernatant, were confirmed by ELISA.

In addition, the expressions of the cartilage metabolic factors MMP3 and MMP13 and the inflammatory factor iNOS were analyzed by qRT-PCR. In addition, the conventional osteoarthritis therapeutic agent Celecoxib was a drug that improved pain and symptoms caused by osteoarthritis. Thus, in order to compare the cartilage regeneration effect with the iPSC-derived mitochondria of the present disclosure, the expression of cartilage regeneration factors SOX9, RUNX2, Aggrecan, and Type II collagen was analyzed by quantitative PCR (qRT-PCR).

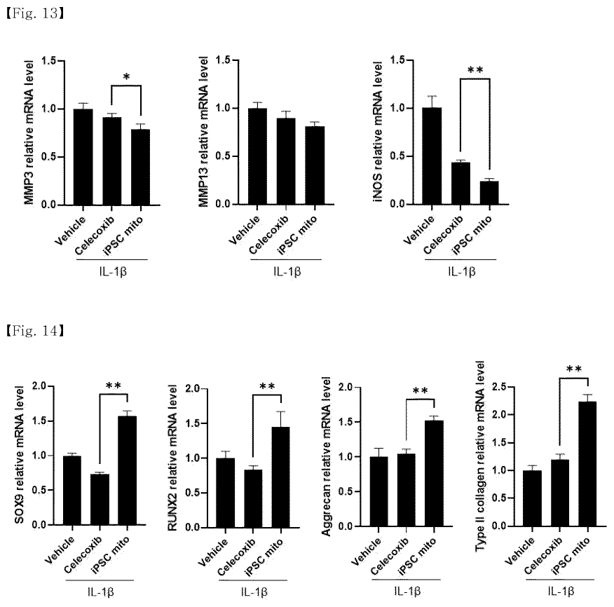

As a result, as shown in FIG. 12, compared to the Celecoxib group, in the iPSC group, the secretion amount of IL-6 was decreased and the secretion amount of IL-8 was significantly decreased, and thus it was confirmed that the iPSC-derived mitochondria of the present disclosure were more effective in regulating inflammatory cytokines than conventional osteoarthritis therapeutic agents. In addition, the cartilage metabolism factors MMP3 and MMP13 and the inflammatory factor iNOS were all reduced in the Celecoxib group and the iPSC mito group compared to the Vehicle group, and the expression of MMP3, MMP13, and iNOS was significantly reduced in the iPSC mito group compared to the Celecoxib group (FIG. 13). Thus, it was confirmed that the iPSC-derived mitochondria of the present disclosure were more effective in regulating inflammation and inhibiting cartilage metabolism than the conventional osteoarthritis therapeutic agent Celecoxib.

In addition, as a result of confirming the expression of cartilage regeneration factors, it was confirmed that the expression of cartilage metabolism regeneration factors in the Celecoxib group was similar to that in the Vehicle group or decreased compared to the Vehicle group, and thus it was confirmed that Celecoxib was a drug that did not promote cartilage regeneration. In contrast, it was confirmed that the iPSC-derived mitochondria of the present disclosure significantly increased the expression of cartilage regeneration factors SOX9, RUNX2, Aggrecan, and Type II collagen (FIG. 14), and thus, it was confirmed that the iPSC-derived mitochondria promoted cartilage regeneration in osteoarthritis to improve the pathogenesis of osteoarthritis.

Therefore, it was confirmed that the iPSC-derived mitochondria of the present disclosure increased the transplant ability into chondrocytes, suppressed the expression of the MMP series, which were cartilage metabolic factors, and suppressed the death of chondrocytes. In addition, it was confirmed that the iPSC-derived mitochondria suppressed damage to joint tissue in an osteoarthritis animal model, reduced osteoarthritis-causing immune cells, and increased immune regulatory cells. In addition, it was confirmed that the expression of UCP2 in iPSC-derived mitochondria was significantly increased compared to mitochondria isolated from other origin cells, and that UCP2 was closely related to a treatment effect of osteoarthritis caused by mitochondrial transplantation. In addition, it was confirmed that iPSC-derived mitochondria overexpressing UCP2 improved osteoarthritis as a cartilage regeneration effect.

1. A method for the preventing or treating osteoarthritis, comprising administering to a subject in need thereof a composition comprising induced pluripotent stem cell (iPSC)-derived mitochondria as an active ingredient.

2. The method of claim 1, wherein the mitochondria are mitochondria that overexpress uncoupling protein 2 (UCP2).

3. The method of claim 1, wherein the mitochondria suppress the expression of cartilage metabolic factors.

4. The method of claim 3, wherein the cartilage metabolic factor is a factor selected from the group consisting of MMP3, MMP9, MMP13, and MCP1.

5. The method of claim 1, wherein the mitochondria suppress the death of chondrocytes.

6. The method of claim 5, wherein the suppressing of the death of chondrocytes is suppressing phosphorylation of RIP3 or MLKL.

7. The method of claim 1, wherein the mitochondria regulate immune cells.

8. The method of claim 7, wherein the immune cell is Th1, Th2, Th17 or Treg.

9. The method of claim 7, wherein the regulating of the immune cells is suppressing Th1, Th2 or Th17.

10. The method of claim 7, wherein the regulating of the immune cells is increasing Treg.

11. The method of claim 1, wherein the mitochondria increase the expression of cartilage regeneration factors.

12. The method of claim 11, wherein the cartilage regeneration factor is selected from the group consisting of transcription factor SOX9 (SOX9), Runt-related transcription factor 2 (RUNX2), Aggrecan, and Type II collagen.

13. A method for cartilage regeneration, comprising administering to a subject in need thereof a composition comprising iPSC-derived mitochondria as an active ingredient.
